(19) United States
(12) Patent Application Publication
(10) Pub. No.: US 2020/0306151 A1
(43) Pub. Date: Oct. 1, 2020

(54) OIL-IN-WATER EMULSION COSMETIC USING ULTRAMICROEMULSION

(71) Applicant: SHISEIDO COMPANY, LTD., TOKYO (JP)

(72) Inventors: Shigeo TAKAHASHI, KANAGAWA (JP); Akane YANAGIHARA, KANAGAWA (JP)

(73) Assignee: SHISEIDO COMPANY, LTD., TOKYO (JP)

(21) Appl. No.: 16/649,398

(22) PCT Filed: Sep. 28, 2018
(86) PCT No.: PCT/JP2018/036536

§ 371 Date: Mar. 20, 2020

(30) Foreign Application Priority Data
Sep. 29, 2017 (JP) 2017-191833

(51) Int. Cl. A61K 8/06; A61K 8/73; A61K 8/92; A61K 8/34; A61K 8/60

(52) U.S. Cl. CPC A61K 8/062; A61K 8/731; A61K 8/922; A61K 2800/5422; A61K 8/60; A61K 2800/21; A61K 2800/412; A61K 8/342

(57) ABSTRACT

An oil-in-water emulsion cosmetic using an ultramicroemulsion that is excellent in transparency and high in viscosity is provided. The oil-in-water emulsion cosmetic includes: (A) 10 to 50% by mass of an ultramicroemulsion including the following (a1) and (a2): (a1) is an anionic surfactant, and (a2) is a linear higher alcohol having 16 or more carbon atoms that can form an α-gel in water in combination with (a1); (B) 0.2 to 2% by mass of a stearoxy hydroxypropyl methyl cellulose having the following formula (1); and (C) 0.1 to 1% by mass of a nonionic surfactant.

### RELATED APPLICATION

This application claims the priority to Japanese Patent Application No. 2017-191833, filed on Sep. 29, 2017, the disclosure of which is incorporated herein by reference in its entirety.

### FIELD OF THE INVENTION

The present invention relates to an oil-in-water emulsion cosmetic using an ultramicroemulsion, and particularly relates to improving transparency and increasing viscosity of the cosmetic.

Hydrophobically modified Ethoxylated URethane Copolymer (HEUR) is also blended to cosmetic compositions and the like as a water-soluble thickener that exhibits excellent viscosity stability and usability (for example, refer to Patent Literature 1). A composition thickened with a hydrophobically modified ethoxylated urethane copolymer gives a unique jiggly texture and is attractive as a cosmetic base to be applied to the skin and the like.

However, although the composition is characterized in that the viscosity is not easily changed by the variation of the co-blended salt concentration or the pH of the composition, there was a problem that the viscosity decreased when it was stored at high temperature (e.g. at 50° C.). Patent Literature 1 indicates that adding a water-soluble polymer such as a carboxyvinyl polymer or xanthan gum in addition to the hydrophobically modified ethoxylated urethane copolymer can suppress the decrease in viscosity at high temperature.

Patent Literatures 2 and 3 describe that a synergistic thickening effect can be achieved by combining a hydrophobically modified ethoxylated urethane copolymer with a microgel of a thickener. In Patent Literature 2, a microgel obtained by pulverizing a gel consisting of a hydrophilic compound having a gelling ability such as agar or gellan gum is blended, and in Patent Literature 3, a microgel obtained by dissolving a water-soluble ethylenically unsaturated monomers (specifically, dimethylacrylamide and 2-acrylamide-2-methylpropanesulfonic acid) in a dispersion phase and radically polymerizing them in the dispersion phase is blended.

When a water-soluble polymer as described in Patent Literature 1 or a microgel of a thickener as described in Patent Literatures 2 and 3 are co-blended to a composition comprising a hydrophobically modified ethoxylated urethane copolymer, an improvement in high-temperature stability and a synergistic thickening effect can be achieved, however, the unique jiggly texture of the composition thickened with a hydrophobically modified ethoxylated urethane copolymer may be lost by mixing a third component such as an oily component or an amphiphilic substance.

Under such circumstances, in Patent Literature 4, an elastic gel composition, in which the high-temperature stability of an oil-in-water emulsion composition thickened with a hydrophobically modified ethoxylated urethane copolymer is improved and the unique jiggly texture is maintained, was developed.

However, the elastic gel composition obtained in Patent Literature 4 did not have a sufficient viscosity. In addition, there was also a problem that even when a thickener generally used in cosmetics was used, the appearance often became cloudy, and conversely, the viscosity was lowered.

### CITATION LIST

### Patent Literatures

### PATENT LITERATURE 1: Japanese Patent No. 3828700 B

### PATENT LITERATURE 2: Japanese Patent No. 4979095 B

### PATENT LITERATURE 3: Japanese Patent No. 5035948 B

### PATENT LITERATURE 4: Japanese Unexamined Patent Publication No. 2016-88868 A

### DISCLOSURE OF THE INVENTION

### Problem to be Solved by the Invention

The present invention has been made in view of the prior arts, and the problem to be solved by the present invention is to provide an oil-in-water emulsion cosmetic having a high viscosity while satisfactorily maintaining a transparent appearance even as a high-pressure emulsion cosmetic.

### Means to Solve the Problem

As a result of diligent study to solve the problem, the present inventors have found that an oil-in-water emulsion cosmetic having a high viscosity while satisfactorily maintaining a transparent appearance can be obtained by blending an ultramicroemulsion, a stearoxy hydroxypropyl methyl cellulose and a nonionic surfactant.

That is, the oil-in-water emulsion cosmetic according to the present invention comprises:

(A) 10 to 50% by mass of an ultramicroemulsion comprising the following (a1) and (a2):

(a1) an anionic surfactant, and

(a2) a linear higher alcohol having 16 or more carbon atoms that can form an α-gel in water in combination with (a1);

(B) 0.2 to 2% by mass of a stearoxy hydroxypropyl methyl cellulose having the following formula (1); and
(C) 0.1 to 1% by mass of a nonionic surfactant.

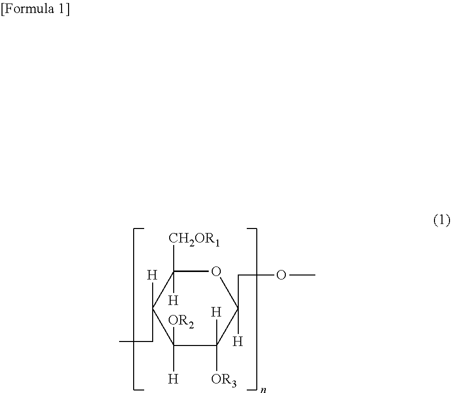

In the formula (1), R₁, R₂ and R₃ are identical or different and represent one or more groups selected from —[CH₂CH(CH₃)O]x-R₄, —[CH₂CH₂O]y-R₄ and —[CH₂CH(OH)CH₂O]z-R₄. Here, x, y, and z are identical or different and represent an integer of 0 to 4.

Moreover, R₄ represents one or more groups selected from a hydrogen atom, a linear or branched alkyl group having 1 to 4 carbon atoms, and a linear alkyl group having 10 to 28 carbon atoms, and at least one of R₄ in the formula (1) is a linear alkyl group having 10 to 28 carbon atoms. The linear alkyl group having 10 to 28 carbon atoms is preferably a linear alkyl group having 12 to 22 carbon atoms.

In the formula (1), n represents an integer of 200 to 200000.

In the oil-in-water emulsion cosmetic, the viscosity is preferably 2000 to 100000 mPa·s.

In the oil-in-water emulsion cosmetic, the ultramicroemulsion (A) is preferably composed of oil droplets having an average particle size of 5 nm to 300 nm.

### Effect of the Invention

According to the present invention, it was found that an oil-in-water emulsion cosmetic having a high viscosity while satisfactorily maintaining its appearance can be obtained by blending an ultramicroemulsion, a specific stearoxy hydroxypropyl methyl cellulose and a nonionic surfactant.

### BEST MODE FOR CARRYING OUT THE INVENTION

The oil-in-water emulsion cosmetic according to the present invention is obtained by blending: (A) an ultramicroemulsion, (B) a stearoxy hydroxypropyl methyl cellulose, and (C) a nonionic surfactant.

The ultramicroemulsion (A) used in the present invention comprises: (a1) an anionic surfactant; and (a2) a linear higher alcohol having 16 or more carbon atoms that can form an α-gel in water in combination with (a1). In general, an α-gel is an aggregate formed by a higher aliphatic alcohol and a hydrophilic surfactant in water, and is a gel having an α-structure (Shoji Fukushima, in “Physical Chemistry of Cetyl Alcohol”, Fragrance Journal Ltd.). The transition temperature of the gel is preferably 60° C. or more in terms of stability.

(A) Ultramicroemulsion

An emulsion refers to an emulsified substance conventionally and widely used in cosmetics and the like.

The ultramicroemulsion used in the present invention is composed of oil droplets having an average particle size of 5 nm to 300 nm. Oil droplets of 10 nm to 150 nm are preferable. If they are larger than 300 nm, the appearance of the composition may change and the aimed transparent appearance of the present invention may not be achieved.

The ultramicroemulsion (A) used in the present invention can be prepared by a method such as an aggregation method or a dispersion method.

An aggregation method is a method for preparing a colloid by utilizing surface chemical properties and is a method of changing a uniformly melted state to a supersaturated state by some means to produce a disperse phase. As specific methods, the HLB temperature emulsification method, the phase inversion emulsification method, the nonaqueous emulsification method, the D-phase emulsification method, the liquid crystal emulsification method, and the like are known.

A dispersion method is a method in which an agglomerate of a disperse phase is turned into fine particles by force. Specifically, it is an emulsification method using the pulverizing force of an emulsifier.

In the present invention, a dispersion method by high-pressure emulsification as described in Japanese Patent No. 3398171 is preferably used. High-pressure emulsification is a method in which an aqueous-phase component and an oil-phase component are pre-emulsified by a homomixer or the like as necessary to obtain an emulsified substance having fine emulsified particles by a high shearing force using a high-pressure homogenizer under high pressure, for example.

When the ultramicroemulsion (A) of the present invention is used in an oil-in-water emulsion cosmetic, the ultramicroemulsion (A) is preferably 10 to 50% by mass in the cosmetic. The ultramicroemulsion (A) is a high-pressure emulsified part composed of a finely emulsified anionic surfactant, an oil component and water.

(a1) Anionic Surfactant

An anionic surfactant that can be used in the present invention is not limited in particular. Examples of anionic surfactants include, but not limited to: fatty acid soap (such as sodium laurate and sodium palmitate); higher alkyl sulfate ester salt (such as sodium lauryl sulfate and potassium lauryl sulfate); alkyl ether sulfate ester salt (such as POE-lauryl sulfate triethanolamine and sodium POE-lauryl sulfate); N-acyl sarcosinic acid (such as sodium lauroyl sarcosinate); higher fatty acid amide sulfonate (such as sodium N-myristoyl-N-methyltaurate, sodium methyl cocoyl taurate and sodium laurylmethyl taurate); phosphate ester salt (sodium POE-oleylether phosphate and POE-stearylether phosphate); sulfosuccinate (such as sodium di-2-ethylhexyl sulfosuccinate, sodium monolauroyl monoethanolamide polyethylene sulfosuccinate and sodium lauryl polypropylene glycol sulfosuccinate); alkylbenzene sulfonate (such as sodium linear dodecylbenzene sulfonate, triethanolamine linear dodeylbenzene sulfonate, and linear dodecylbenzene sulfonate); higher fatty acid ester sulfate ester salt (such as sodium hydrogenated gryceryl cocoate sulfate); N-acyl glutamate (such as monosodium N-lauroyl glutamate, disodium N-stearoyl glutamate and monosodium N-myristoyl-L-glutamate); sulfonated oil (such as Turkey red oil); POE-alkyl ether carboxylic acid; POE-alkyl aryl ether carboxylate; α-olefin sulfonate; higher fatty acid ester sulfonate; secondary alcohol sulfate ester salt; higher fatty acid alkylolamide sulfate ester salt; sodium lauroyl monoethanolamide succinate; N-palmitoyl asparaginate ditriethanolamine; and sodium casein.

Among the above, long-chain acyl sulfonate anionic surfactants, N-long-chain acyl acidic amino acid salts, soaps and the like are preferred.

Sodium stearoyl methyl taurate, sodium N-stearoyl-L-glutamate, sodium N-lauroyl-L-glutamate, potassium N-myristoyl-L-glutamate, stearic acid, lauric acid, behenic acid and the like are more preferred.

Examples of commercially available products of the anionic surfactant (a1) include, but not limited to: sodium stearoyl glutamate (AMISOFT HS-11P(F), manufactured by Ajinomoto Co., Inc.), sodium lauroyl glutamate (AMISOFT LS-11, manufactured by Ajinomoto Co., Inc.), potassium myristoyl glutamate (AMISOFT MK-11, Ajinomoto Co., Inc.), and sodium myristoyl glutamate (AMISOFT MS-11, manufactured by Ajinomoto Co., Inc.).

(a2) Linear Higher Alcohol Having 16 or More Carbon Atoms

A linear higher alcohol having 16 or more carbon atoms (a2) is not particularly limited as long as it can form an α-gel in water in combination with (a1). Examples of linear higher alcohols having 16 or more carbon atoms include, but not limited to: linear and branched higher alcohols such as lauryl alcohol, cetyl alcohol, stearyl alcohol, behenyl alcohol, myristyl alcohol, oleyl alcohol, cetostearyl alcohol, monostearyl glycerin ether (batyl alcohol), 2-decyltetradecinol, lanolin alcohol, cholesterol, hexyldodecanol, isostearyl alcohol and octyldodecanol.

The stearoxy hydroxypropyl methyl cellulose (B) used in the present invention is represented by the following formula (1).

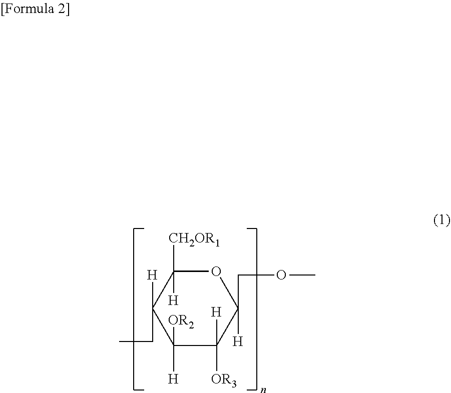

In the formula (1), R₁, R₂ and R₃ are identical or different and represent one or more groups selected from —[CH₂CH(CH₃)O]x-R₄, —[CH₂CH₂O]y-R₄ and —[CH₂CH(OH)CH₂O]z-R₄. Here, x, y, and z are identical or different and represent an integer of 0 to 4.

Moreover, R₄ represents one or more groups selected from a hydrogen atom, a linear or branched alkyl group having 1 to 4 carbon atoms, and a linear alkyl group having 10 to 28 carbon atoms, and at least one of R₄ in the formula (1) is a linear alkyl group having 10 to 28 carbon atoms. The linear alkyl group having 10 to 28 carbon atoms is preferably a linear alkyl group having 12 to 22 carbon atoms.

In the formula (1), n represents an integer of 200 to 200000.

Commercial products of the stearoxy hydroxypropyl methyl cellulose (B) are available from Daido Chemical Corporation under the trade name SANGELOSE, and examples thereof include, but not limited to, “SANGELOSE 90L”, “SANGELOSE 90M”, and “SANGELOSE 60L”.

When the stearoxy hydroxypropyl methyl cellulose (B) used in the present invention is used in an oil-in-water emulsion cosmetic, it can be blended to the cosmetic in an amount of 0.2 to 2% by mass, and it is further preferable to blend it in an amount of 0.3 to 1%.

(C) Nonionic Surfactant

Examples of nonionic surfactants that can be used in the present invention include, but not limited to: polyoxyethylene fatty acid glyceryl, polyoxyethylene methylpolysiloxane copolymer, fatty acid polyoxyethylene sorbitan, polyoxyethylene alkyl ether, maltitol hydroxy aliphatic alkyl ether, alkylated polysaccharide, alkyl glucoside, sucrose fatty acid ester, polyoxyethylene hydrogenated castor oil glyceryl, polyoxyethylene cetyl ether, polyoxyethylene hydrogenated castor oil, polyoxyethylene glyceryl isostearate N, polyoxyethylene phytosterol, polyoxyethylene behenyl ether and polyoxyethylene sorbitan monostearate.

Among the above, those having an HLB of 8 or more are particularly preferred.

Examples of commercial products of the nonionic surfactant (C) used in the present invention include, but not limited to: NIKKOL® HCO-60 (manufactured by Nikko Chemicals Co., Ltd.), EMALEX® GWIS-160N (manufactured by Nihon Emulsion Co., Ltd.), NIKKOL® BPS-30 (manufactured by Nikko Chemicals Co., Ltd.), NIKKOL® BB-20 (manufactured by Nikko Chemicals Co., Ltd.), NIKKOL® TS-10V (manufactured by Nikko Chemicals Co., Ltd.), sorbitan laurate ((NIKKOL® SL-10 and LM, Nikko Chemicals Co., Ltd.); (EMASOL® 110, Kao Corporation)), sorbitan palmitate (NIKKOL® SP-10, Nikko Chemicals Co., Ltd.), sorbitan oleate (NIKKOL® SO-10V, Nikko Chemicals Co., Ltd.), sorbitan stearate (NIKKOL® SS-10, Nikko Chemicals Co., Ltd.), sorbitan isostearate (NIKKOL® SI-10PKV, Nikko Chemicals Co., Ltd.), sorbitan sesquioleate ((NIKKOL® SO-15, Nikko Chemicals Co., Ltd.); (RHEODOL® AO-15, Kao Corporation); (NOFABLE® SO-852S, NOF Corporation), sorbitan trioleate ((RHEODOL® SP-O-30, Kao Corporation); (NOFABLE® SO-853S, NOF Corporation), sorbitan tristearate (NIKKOL® SS-30V, Nikko Chemicals Co., Ltd.), sorbitan sesquiisostearate (ESTEMOL® 182V, Nisshin OilliO Group, Ltd.), Polysorbate 60 ((Tween® 60, Croda International Plc); (NIKKOL® TS-10V, Nikko Chemicals Co., Ltd.)), Polysorbate 80 ((RHEODOL® TWO-120, Kao Corporation); (NIKKOL® TO-10V, Nikko Chemicals Co., Ltd.)).

PEG-5 glyceryl stearate (EMALEX® GWIS GM-5, Nihon Emulsion Co., Ltd.), PEG-20 glyceryl stearate (POEM S-120, PEG-8 glyceryl isostearate (EMALEX® GWIS-108, Nihon Emulsion Co., Ltd.), PEG-20 glyceryl isostearate (EMALEX® GWIS-120, Nihon Emulsion Co., Ltd.), PEG-60 glyceryl isostearate (EMALEX® GWIS-160N, Nihon Emulsion Co., Ltd.), PEG-90 glyceryl isostearate (EMALEX® GWIS-190, Nihon Emulsion Co., Ltd.), PEG-5 glyceryl triisostearate (EMALEX® GWIS-305, Nihon Emulsion Co., Ltd.), PEG-20 glyceryl triisostearate (EMALEX® GWIS-320EX, Nihon Emulsion Co., Ltd.), PEG-20 glyceryl triisostearate (EMALEX® GWIS-320, Nihon Emulsion Co., Ltd.), PEG-20 hydrogenated castor oil isostearate (EMALEX® RWIS-120, Nihon Emulsion Co., Ltd.), PEG-5 glyceryl triisostearate (EMALEX® RWIS-305, Nihon Emulsion Co., Ltd.), glyceryl stearate (SE) (SUNSOFT® 30, Taiyo Kagaku Co., Ltd.), glyceryl stearate (SUNSOFT® 8004, Taiyo Kagaku Co., Ltd.); (NIKKOL® MGS-EEXV, Nikko Chemicals Co., Ltd.)), glyceryl oleate ((SUNSOFT® 301V and O-30S, Taiyo Kagaku Co., Ltd.), (RHEODOL® MO-60, Kao Corporation)), glyceryl cottonseed fatty acid (SUNSOFT® 8080, Taiyo Kagaku Co., Ltd.), and the like can be included.

When the nonionic surfactant (C) used in the present invention is used in an oil-in-water emulsion cosmetic, the nonionic surfactant (C) is preferably 0.1 to 1% by mass in the cosmetic. If it exceeds 1%, it may not be preferable in that gelation tends to occur high temperature. If it is less than 0.1%, it may not be preferable in that the viscosity required in the present invention cannot achieved, and thus separation tends to occur.

It was found that when the blending ratio of the nonionic surfactant (C) with respect to the stearoxy hydroxypropyl methyl cellulose (B) is within the range of 1:0.3 to 1:4, an oil-in-water emulsion cosmetic having a high viscosity while satisfactorily maintaining its appearance can be obtained. If the ratio exceeds 1:4, it may not be preferable in that the gelling tendency becomes stronger. If the ratio is less than 1:0.3, it may not be preferable in that it tends to separate due to a decrease in viscosity.

In the oil-in-water emulsion cosmetic obtained by blending the ultramicroemulsion (A), the stearoxy hydroxypropyl methyl cellulose (B), and the nonionic surfactant (C), the viscosity is preferably 2000 to 100000 mPa·s. It is more preferable when the viscosity is 2000 to 10000 mPa·s. In such a viscosity range, the cosmetic is a gel when placed on the skin, but it immediately changes into a liquid and provides a fresh feeling when a shear is applied thereto.

The oil-in-water emulsion cosmetic of the present invention is prepared by blending various components for making a cosmetic into the oil-in-water emulsion cosmetic. The other various components are blended into the aqueous phase (continuous phase) or the oil phase (disperse phase) of the oil-in-water emulsion in accordance with their properties.

### [Other Components]

The cosmetic that uses the cosmetic raw material according to the present invention may include, as appropriate and as necessary, other components within the range of not inhibiting the effect of the present invention such as inorganic powders, organic powders, esters, anionic surfactants, cationic surfactants, amphoteric surfactants, nonionic surfactants, moisturizers, water-soluble polymers, thickeners, film-forming agents, ultraviolet-light absorbers, sequestrants, lower alcohols, polyhydric alcohols, saccharides, amino acids, organic amines, polymeric emulsions, pH adjusters, skin nutrients, vitamins, antioxidants, antioxidant aids, perfumes, water and the like, and can be produced by a usual manner in accordance with the aimed form.

Examples of inorganic powders include, but not limited to: talc, boron nitride, sericite, natural mica, fired mica, synthetic mica, synthetic sericite, alumina, mica, kaolin, bentonite, smectite, calcium carbonate, magnesium carbonate, calcium phosphate, silicic anhydride, magnesium oxide, tin oxide, iron oxide, yttrium oxide, chromium oxide, zinc oxide, cerium oxide, aluminum oxide, magnesium oxide, chromium hydroxide, iron blue, ultramarine, calcium phosphate, aluminum hydroxide, barium sulfate, magnesium sulfate, silicic acid, aluminum magnesium silicate, calcium silicate, barium silicate, magnesium silicate, aluminum silicate, strontium silicate, silicon carbide, magnesium fluoride, tungstate, magnesium aluminate, magnesium aluminometasilicate, chlorohydroxyaluminum, clay, zeolite, hydroxyapatite, ceramic powder, spinel, mullite, cordierite, aluminum nitride, titanium nitride, silicon nitride, lanthanum, samarium, tantalum, terbium, europium, neodymium, Mn—Zn ferrite, Ni—Zn ferrite, silicon carbide, cobalt titanate, barium titanate, iron titanate, lithium cobalt titanate, cobalt aluminate, antimony-containing tin oxide, tin-containing indium oxide, magnetite, aluminum powder, gold powder, silver powder, platinum powder, copper powder, precious metal colloid, iron powder, zinc powder, cobalt blue, cobalt violet, cobalt green, lower titanium oxide, particulate titanium oxide, butterfly-like barium sulfate, petal-like zinc oxide, tetrapod-like zinc oxide, particulate zinc oxide, pearl pigments such as titanium oxide-coated mica, titanium oxide-coated mica, titanium oxide-coated synthetic mica, titanium oxide-coated silica, titanium oxide-coated synthetic mica, titanium oxide-coated talc, zinc oxide-coated silica, titanium oxide-coated colored mica, red iron oxide-coated mica titanium, red iron oxide/black iron oxide-coated mica titanium, carmine-coated mica titanium, and iron blue-coated mica titanium.

Examples of organic powders include, but not limited to: silicone elastomer powder, silicone powder, silicone resin coated silicone elastomer powder, polyamide resin powder (nylon powder), polyethylene powder, polymethyl methacrylate powder (e.g. methyl methacrylate crosspolymer), polystyrene powder, styrene-acrylic acid copolymer powder, benzoguanamine resin powder, poly(tetrafluoroethylene) powder, and cellulose powder. Examples of organic pigments include, but not limited to: zirconium, barium, or aluminum lake (such as organic pigment such as Red No. 201, Red No. 202, Red No. 204, Red No. 205, Red No. 220, Red No. 226, Red No. 228, Red No. 405, Orange No. 203, Orange No. 204, Yellow No. 205, Yellow No. 401, and Blue No. 404).

Examples of anionic surfactants include, but not limited to: fatty acid soap (such as sodium laurate, and sodium palmitate); higher alkyl sulfate ester salt (such as sodium lauryl sulfate, and potassium lauryl sulfate); alkyl ether sulfate ester salt (such as POE-lauryl sulfate triethanolamine, and sodium POE-lauryl sulfate); N-acyl sarcosinic acid (such as sodium lauroyl sarcosinate); higher fatty acid amide sulfonate (such as sodium N-myristoyl-N-methyltaurate, sodium methyl cocoyl taurate, and sodium laurylmethyl taurate); phosphate ester salt (sodium POE-oleylether phosphate, and POE-stearylether phosphate); sulfosuccinate (such as sodium di-2-ethylhexyl sulfosuccinate, sodium monolauroyl monoethanolamide polyethylene sulfosuccinate, and sodium lauryl polypropylene glycol sulfosuccinate); alkylbenzene sulfonate (such as sodium linear dodecylbenzene sulfonate, triethanolamine linear dodeylbenzene sulfonate, and linear dodecylbenzene sulfonate); higher fatty acid ester sulfate ester salt (such as sodium hydrogenated gryceryl cocoate sulfate); N-acyl glutamate (such as monosodium N-lauroyl glutamate, disodium N-stearoyl glutamate, and monosodium N-myristoyl-L-glutamate); sulfonated oil (such as Turkey red oil); POE-alkyl ether carboxylic acid; POE-alkyl aryl ether carboxylate; α-olefine sulfonate; higher fatty acid ester sulfonate; secondary alcohol sulfate ester salt; higher fatty acid alkylolamide sulfate ester salt; sodium lauroyl monoethanolamide succinate; N-palmitoyl asparaginate ditriethanolamine; and sodium casein.

Examples of cationic surfactants include, but not limited to: alkyltrimethyl ammonium salt (such as stearyltrimethyl ammonium chloride, lauryltrimethyl ammonium chloride); alkylpyridinium salt (such as cetylpyridinium chloride); distearyldimethyl ammonium chloride; dialkyldimethyl ammonium salt; poly (N,N′-dimethyl-3,5-methylenepiperidinium) chloride; alkyl quaternary ammonium salt; alkyldimethylbenzyl ammonium salt; alkylisoquinolinium salt; dialkylmorphonium salt; POE-alkylamine; alkylamine salt; polyamine fatty acid derivative; amyl alcohol fatty acid derivative; benzalkonium chloride; and benzethonium chloride.

Examples of amphoteric surfactant include, but not limited to: imidazoline-based amphoteric surfactant (such as sodium 2-undecyl-N,N,N-(hydroxyethylcarboxymethyl)-2-imidazoline and 2-cocoyl-2-imidazolinium hydroxide-1-carboxyethyloxy disodium salt); and betaine-based surfactant (such as 2-heptadecyl-N-carboxymethyl-N-hydroxyethyl imidazolinium betaine, lauryl dimethylaminoacetic acid betaine, alkyl betaine, amidobetaine, and sulfobetaine).

Examples of lipophilic nonionic surfactants include, but not limited to: sorbitan fatty acid esters (such as sorbitan monooleate, sorbitan monoisostearate, sorbitan monolaurate, sorbitan monopalmitate, sorbitan monostearate, sorbitan sesquioleate, sorbitan trioleate, diglycerol sorbitan penta-2 ethylhexylate, diglycerol sorbitan tetra-2 ethylhexylate, etc.); glyceryl polyglyceryl fatty acids (such as glyceryl monocotton oil fatty acid, glyceryl monoerucate, glyceryl sesquioleate, glyceryl monostearate, glyceryl α, α′-oleate pyroglutamate, glyceryl monostearate malate, etc.); propylene glycol fatty acid esters (such as propylene glycol monostearate, etc.); hydrogenated castor oil derivative; and glyceryl alkyl ether.

Examples of hydrophilic nonionic surfactants include, but not limited to: POE sorbitan fatty acid ester (such as POE sorbitan monooleate, POE sorbitan monostearate, POE sorbitan monooleate, POE sorbitan tetraoleate); POE sorbit fatty acid ester (such as POE sorbit monolaurate, POE sorbit monooleate, POE sorbit pentaoleate, POE sorbit monostearate), POE glyceryl fatty acid ester (such as POE monooleate such as POE glyceryl monostearate, POE glyceryl monoisostearate, POE glyceryl triisostearate); POE fatty acid ester (such as POE distearate, POE monodioleate, ethyleneglycol distearate); POE alkyl ether (such as POE lauryl ether, POE oleyl ether, POE stearyl ether, POE behenyl ether, POE-2-octyldodecyl ether, POE cholestanol ether); puluronic type (such as Puluronic), POE/POP alkyl ethers (such as POE/POP cetyl ether, POE/POP 2-decyltetradecyl ether, POE/POP monobutyl ether, POE/POP hydrogenated lanoline, POE/POP glycerin ether); tetra POE/tetra POP ethylenediamine condensation products (such as Tetronic); POE castor oil hydrogenated castor oil derivative (such as POE castor oil, POE hydrogenated castor oil, POE hydrogenated castor oil monoisostearate, POE hydrogenated castor oil triisostearate, POE hydrogenated castor oil monopyroglutamate monoisostearate diester, POE hydrogenated oil maleate); POE beeswax/lanoline derivative (such as POE sorbitol beeswax); alkanolamide (such as coconut oil fatty acid diethanolamide, lauric acid monoethanolamide, fatty acid isopropanolamide); POE propyleneglycol fatty acid ester; POE alkyl amines; POE fatty acid amide; sucrose fatty acid ester; alkylethoxydimethylamine oxide; and trioleyl phosphoric acid.

Examples of moisturizers include, but not limited to: polyethylene glycol, propylene glycol, glycerin, 1,3-butylene glycol, xylitol, sorbitol, maltitol, chondroitin sulfate, hyaluronic acid, mucoitin sulfate, charonic acid, atelocollagen, cholesteryl 12-hydroxystearate, sodium lactate, bile salt, dl-pyrrolidone carboxylate, alkyleneoxide derivative, short-chain soluble collagen, diglycerin (EO)PO adduct, chestnut rose extract, yarrow extract, and melilot extract.

Examples of natural water-soluble polymers include, but not limited to: plant-based polymers (such as gum Arabic, gum tragacanth, galactan, guar gum, locust bean gum, gum karaya, carrageenan, pectine, agar, quince seed (Cydonia oblonga), algae colloid (brown algae extract), starch (rice, corn, potato, wheat), glicyrrhizic acid); microorganism-based polymers (such as xanthan gum, dextran, succinoglycan, pullulan, etc.); and animal-based polymers (such as collagen, casein, albumin, gelatin, etc.).

Examples of semisynthetic water-soluble polymers include, but not limited to: starch-based polymers (such as carboxymethyl starch, methylhydroxypropyl starch, etc.); cellulose-based polymers (such as methylcellulose, ethylcellulose, methylhydroxypropylcellulose, hydroxyethylcellulose, cellulose sodium sulfate, hydroxypropylcellulose, carboxymethylcellulose, sodium calboxymethyl cellulose, crystalline cellulose, cellulose powder, etc.); and algin acid-based polymers (such as sodium alginate, propylene glycol alginate ester, etc.).

Examples of synthetic water-soluble polymers include, but not limited to: vinyl-based polymers (such as polyvinyl alcohol, polyvinyl methyl ether, polyvinylpyrrolidone, carboxyvinylpolymer, etc.); polyoxyethylene-based polymers (such as polyoxyethylenepolyoxypropylene copolymer such as polyethylene glycol 20,000, 40,000 and 60,0000, etc.); acrylic polymers (such as sodium polyacrylate, polyethylacrylate, polyacrylamide, etc.); polyethyleneimine; and cationic polymer.

Examples of thickeners include, but not limited to: gum arabic, carrageenan, karaya gum, tragacanth gum, carob gum, quince seed (marmelo), casein, dextrin, gelatin, sodium pectate, sodium alginate, methylcellulose, ethyl cellulose, CMC, hydroxyethyl cellulose, hydroxypropyl cellulose, PVA, PVM, PVP, sodium polyacrylate, carboxyvinyl polymer, locustbean gum, guar gum, tamarind gum, dialkyldimethylammonium sulfate cellulose, xanthan gum, magnesium aluminum silicate, bentonite, hectorite, magnesium aluminum silicate (veegum), laponite, and silicic anhydride.

Examples of ultraviolet-light absorbers include, but not limited to: benzoic acid family ultraviolet light absorber (such as p-aminobenzoic acid (hereinafter abbreviated as PABA), PABA monoglycerine ester, N,N-dipropoxy PABA ethyl ester, N,N-diethoxy PABA ethyl ester, N,N-dimethyl PABA ethyl ester, N,N-dimethyl PABA butyl ester, N,N-dimethyl PABA ethyl ester, etc); anthranilic acid family ultraviolet light absorber (such as homomenthyl N-acetylanthranilate etc); salicylic acid family ultraviolet light absorber (such as amyl salicylate, menthyl salicylate, homomenthyl salicylate, octyl salicylate, phenyl salicylate, benzyl salicylate, p-isopropanolphenyl salicylate, etc); cinnamic acid family ultraviolet light absorber (such as octyl methoxycinnamate, ethyl 4-isopropylcinnamate, methyl 2,5-diisopropylcinnamate, ethyl 2,4-diisopropylcinnamate, methyl 2,4-diisopropylcinnamate, propyl p-methoxycinnamate, isopropyl p-methoxycinnamate, isoamyl p-methoxycinnamate, octyl p-methoxycinnamate (2-ethylhexyl p-methoxycinnamate), 2-ethoxyethyl p-methoxycinnamate, cyclohexyl p-methoxycinnamate, ethyl α-cyano-J3-phenylcinnamate, 2-ethylhexyl α-cyano-3-phenylcinnamate, glyceryl mono-2-ethylhexanoyl-diparamethoxy cinnamate, etc); benzophenone family ultraviolet light absorber (such as 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl-4′-phenyl-benzophenone-2-carboxylate, 2-hydroxy-4-n-octoxybenzophenone, 4-hydroxy-3-carboxybenzophenone, etc); 3-(4′-methylbenzylidene)-d,l-camphor and 3-benzylidene-d,l-camphor; 2-phenyl-5-methylbenzoxazol; 2,2′-hydroxy-5-methylphenylbenzotriazol, 2-(2′-hydroxy-5′-t-octylphenyl) benzotriazol, 2-(2′-hydroxy-5′-methylphenylbenzotriazol; dibenzalazine; dianisoylmethane; 4-methoxy-4′-t-butyldibenzoylmethane; 5-(3,3-dimethyl-2-norbornylidene)-3-pentane-2-one; dimorpholinopyridazinone; 2-ethylhexyl 2-cyano-3,3-diphenylacrylate; and 2,4-bis-{[4-(2-ethylhexyloxy)-2-hydroxy]-phenyl}-6-(4-methoxyphenyl)-(1,3,5)-triazine.

Examples of sequestrants include, but not limited to: 1-hydroxyethane-1, 1-diphosphonic acid, I-hydroxyethane, 1-diphosphonic acid 4Na salt, disodium edetate, trisodium edetate, tetrasodium edetate, sodium citrate, sodium polyphosphate, sodium metaphosphate, gluconic acid, phosphoric acid, citric acid, ascorbic acid, succinic acid, edetic acid, and trisodium hydroxyethyl ethylenediamine triacetate.

Examples of lower alcohol include, but not limited to: ethanol, propanol, isopropanol, isobutyl alcohol, and t-butyl alcohol.

Examples of polyhydric alcohols include, but not limited to: dihydric alcohol (such as ethylene glycol, propylen glycol, trimethylene glycol, 1,2-butylene glycol, 1,3-butylene glycol, tetramethylene glycol, 2,3-butylene glycol, pentamethylene glycol, 2-butene-1,4-diol, hexylene glycol, octylene glycol, etc); trihydric alcohol (such as glycerin, trimethylolpropane, etc); tetrahydric alcohol (such as such as pentaerythritol such as 1,2,6-hexanetriol, etc); pentahydric alcohol (such as xylitol, etc); hexahydric alcohol (such as sorbitol, mannitol, etc); polyhydric alcohol polymer (such as diethylene glycol, dipropylene glycol, triethylene glycol, polypropylene glycol, tetraethylene glycol, diglycerin, polyethylene glycol, triglycerin, tetraglycerin, polyglycerin, etc); dihydric alcohol alkyl ethers (such as ethylene glycol monomethyl ether, ethylene glycol monoethyl ether, ethylene glycol monobutyl ether, ethylene glycol monomphenyl ether, ethylene glycol monohexyl ether, ethylene glycol mono2-methylhexyl ether, ethylene glycol isoamyl ether, ethylene glycol benzil ether, ethylene glycol isopropyl ether, ethylene glycol dimethyl ether, ethylene glycol diethyl ether, ethylene glycol dibutyl ether, etc); dihydric alcohol alkyl ethers (such as diethylene glycol monomethyl ether, diethylene glycol monoethyl ether, diethylene glycol monombutyl ether, diethylene glycol dimethyl ether, diethylene glycol diethyl ether, diethylene glycol butyl ether, diethylene glycol methylethyl ether, triethylene glycol monomethyl ether, triethylene glycol monoethyl ether, propylene glycol monomethyl ether, propylene glycol monoethyl ether, propylene glycol monobutyl ether, propylene glycol isopropyl ether, dipropylene glycol methyl ether, dipropylene glycol ethyl ether, dipropylene glycol butyl ether, etc); dihydric alcohol ether ethers (such as ethylene glycol monomethyl ether acetate, ethylene glycol monoethyl ether acetate, ethylene glycol monobutyl ether acetate, ethylene glycol monophenyl ether acetate, ethylene glycol diadipate, ethylene glycol disaccinate, diethylene glycol monoethyl ether acetate, diethylene glycol monobutyl ether acetate, propylene glycol monomethyl ether acetate, propylene glycol monoethyl ether acetate, propylene glycol monopropyl ether acetate, propylene glycol monophenyl ether acetate, etc); glycerin monoalkyl ether (such as chimyl alcohol, selachyl alcohol, batyl alcohol, etc); sugar alcohol (such as sorbitol, maltitol, maltotriose, mannitol, sucrose, erythritol, glucose, fructose, starch sugar, maltose, xylitose, starch sugar hydrogenated alcohol, etc); glycolide, tetrahydrofurfuryl alcohol; POE-tetrahydrofurfuryl alcohol; POP/POE-butyl ether; tripolyoxypropylene glycerin ether; POP-glycerin ether; POP-glycerin ether phosphoric acid; POP/POE-pentaerythritol ether; and polyglycerin.

Examples of monosaccharides include, but not limited to: triose (such as D-glyceryl aldehyde, dihydroxyacetone, etc.); tetrose (such as D-erythrose, D-erythrulose, D-threose, erythritol, etc.); pentaose (such as L-arabinose, D-xylose, L-lyxose, D-arabinose, D-ribose, D-ribulose, D-xylulose, L-xylulose, etc.); hexalose (such as D-glucose, D-talose, D-psicose, D-galactose, D-fructose, L-galactose, L-mannose, D-tagatose, etc.); heptose (such as aldoheptose, heplose); octose (such as octulose, etc.); deoxy sugar (such as 2-deoxy-D-ribose, 6-deoxy-L-galactose, 6-deoxy-L-mannose, etc.); amino sugar (such as D-glucosamine, D-galactosamine, sialic acid, amino uronic acid, muramic acid, etc.); uronic acid (such as D-grucuronic acid, D-mannuronic acid, L-guluronic acid, D-garacturonic acid, and L-iduronic acid, etc.).

Examples of oligosaccharides include, but not limited to: sucrose, guntianose, umbelliferose, lactose, planteose, isolignoses, α,α-trehalose, raffinose, lignoses, umbilicin, and stachyoseverbascoses.

Examples of polysaccharides include, but not limited to: cellulose, quince seed, chondroitinsulfate, starch, galactan, dermatan sulfate, glycogen, acasia gum, heparansulfate, hyaluronan, gum tragacanth, keratan sulfate, chondoroitin, xanthan gum, mucoitin sulfate, guar gum, dextran, keratosulfate, locust bean gum, succinoglycan, and caronic acid.

Examples of amino acids include, but not limited to: neutral amino acid (such as threonine, cysteine, etc.); basic amino acid (such as hydroxylysine, etc.) and the like. Examples of the amino acid derivative may include sodium acyl sarcosinate (sodium lauroyl sarcosinate), acyl glutamate, sodium acyl 3-alanine, glutathione, and pyrrolidone carboxylate.

Examples of organic amines include, but not limited to: monoethanolamine, diethanolamine, triethanolamine, morpholine, triisopropanolamine, 2-amino-2-methyl-1,3-propanediol, and 2-amino-2-methyl-1-propanol.

Examples of polymer emulsions include, but not limited to acrylic resin emulsion, ethyl polyacrylate emulsion, solution of acrylic resin, polyacrylalkylester emulsion, polyvinyl acetate resin emulsion, and natural rubber latex.

Examples of pH adjusters include, but not limited to buffers such as lactic acid-sodium lactate, citric acid-sodium citrate, and succinic acid-sodium succinate.

Examples of vitamins include, but not limited to vitamin A, B1, B2, B6, C, E and derivatives thereof, pantothenic acid and derivatives thereof, and biotin.

Examples of anti-oxidants include, but not limited to tocopherols, dibutyl hydroxy toluene, butyl hydroxy anisole, and gallic acid esters.

Examples of anti-oxidant aids include, but not limited to phosphoric acid, citric acid, ascorbic acid, maleic acid, malonic acid, succinic acid, fumaric acid, cephalin, hexamethaphosphate, phytic acid, and ethylenediaminetetraacetic acid.

Examples of other components that can be blended include, but not limited to: an antiseptic agent (such as ethylparaben, butylparaben, chlorphenesin, 2-phenoxyethanol, etc.); antiphlogistic (such as glycyrrhizinic acid derivatives, glycyrrhetic acid derivatives, salicylic acid derivatives, hinokitiol, zinc oxide, allantoin, etc.); a skin-whitening agent (such as placental extract, saxifrage extract, arbutin, etc.); various extracts (such as phellodendron bark (cork tree bark), coptis rhizome, lithospermum, peony, swertia herb, birch, sage, loquat, carrot, aloe, mallow, iris, grape, coix seed, sponge gourd, lily, saffron, cnidium rhizome, ginger, hypericum, restharrow, garlic, red pepper, citrus unshiu, Japanese angelica, seaweed etc.); an activator (such as royal jelly, photosenstizer, cholesterol derivatives, etc.); a blood circulation accelerator (such as nonylic acid vanillylamide, nicotine acid benzyl ester, nicotine acid β-butoxyethyl ester, capsaicin, zingerone, cantharides tincture, ichthammol, tannic acid, α-borneol, tocopheryl nicotinate, meso-inositol hexanicotinate, cyclandelate, cinnarizine, tolazoline, acetylcholine, verapamil, cepharanthine, γ-oryzanol, etc.); an antiseborrheric agent, (such as sulfur, thianthl, etc.); and an anti-inflammatory agent (such as tranexamic acid, thiotaurine, hypotaurine, etc.).

Furthermore, the cosmetic of the present invention can comprise, as appropriate and as necessary: sequestrants such as disodium edetate, trisodium edetate, sodium citrate, sodium polyphosphate, sodium metaphosphate, gluconic acid, malic acid, and the like; caffeine; tannin; verapamil; tranexamic acid and derivatives thereof; various crude drug extracts such as licorice, Chinese quince, Pyrola japonica and the like; drugs such as tocopherol acetate, glycyrrhetinic acid, glycyrrhizic acid and derivatives thereof, or salts thereof; skin-whitening agents such as vitamin C, magnesium ascorbyl phosphate, ascorbic acid glucoside, arbutin, kojic acid and the like; amino acids such as arginine and lysine and the like and derivatives thereof; and saccharides such as fructose, mannose, erythritol, trehalose, xylitol and the like.

The oil-in-water emulsion cosmetic according to the present invention can be made into any product form. Examples of specific product forms thereof include skin care cosmetics such as: moisturizing gels; massage gels; serums; lotions and emulsions; makeup cosmetics; sun care products; hair cosmetics such as hair setting agents and hair gels; and hair dyes.

[Method for Producing the Oil-in-Water Emulsion Cosmetic According to the Present Invention]

The oil-in-water emulsion cosmetic according to the present invention can be produced by a well-known method. For example, a stearoxy hydroxypropyl methyl cellulose and a nonionic surfactant are dissolved in water (A). A part of water, oil and an anionic surfactant are subjected to high-pressure emulsification to give an ultramicroemulsion (B).

By mixing (A) and (B), the target oil-in-water emulsion cosmetic having an appropriate viscosity range can be produced.

### Examples

The present invention is further described in detail below with the examples, but the present invention is not limited thereto. The blending amount is shown in “% by mass” based on the system into which the component is blended, unless otherwise specified.

Before describing the examples, evaluation methods of the oil-in-water emulsion cosmetic according to the present invention are described.

Evaluation (1): Evaluation of Transparency

Transparency is assessed by measuring the L-value of the ultramicroemulsion part and the final appearance. The ultramicroemulsion part is measured with Spectrophotometer SE700 manufactured by NIPPON DENSHOKU to confirm that the L-value is between 30 to 90. In addition, the appearance is assessed based on whether the background can be seen through when it is put in a 50 g glass container.

A: The background can be seen through

B: The background cannot be seen through

Evaluation (2): Evaluation of Viscosity

The viscosity (mPa·s/30° C.) is measured with DIGITAL VISMETRON VDA manufactured by SHIBAURA SYSTEMS CO., LTD.

As the measurement conditions, a rotor No. 3 or 4 was used, and the measurements values were taken at 12 rpm/minute.

The measurement of the viscosity is

Grade A: 2000 to less than 10,000 mPa·s
Grade B: less than 2000 mPa·s, or greater than 10,000 mPa·s
Grade C: impossible to measure

First, the present inventors produced the ultramicroemulsion used in the present invention, and investigated whether the ultramicroemulsion (A) having an average emulsified particle size of 5 nm to 300 nm can be thickened with a commonly used thickener and the nonionic surfactant (C).

TABLE 1

| | | Test Examples | | | | | | | |
|---|---|---|---|---|---|---|---|---|---|
| | | 1-1 | 1-2 | 1-3 | 1-4 | 1-5 | 1-6 | 1-7 | 1-8 |
| (A) | Ion-exchanged water | To 100 | To 100 | To 100 | To 100 | To 100 | To 100 | To 100 | To 100 |
| | Glycerin | 8 | 8 | 8 | 8 | 8 | 8 | 8 | 8 |
| | 1,3-Butylene glycol | 4 | 4 | 4 | 4 | 4 | 4 | 4 | 4 |
| | Sodium stearoyl glutamate | 0.3 | 0.3 | 0.3 | 0.3 | 0.3 | 0.3 | 0.3 | 0.3 |
| | Potassium hydroxide | Suitable | Suitable | Suitable | Suitable | Suitable | Suitable | Suitable | Suitable |
| | | amount | amount | amount | amount | amount | amount | amount | amount |
| | Hydrogenated polyisobutene (*1) | 3 | 3 | 3 | 3 | 3 | 3 | 3 | 3 |
| | Pentaerythrityl tetraethylhexanoate | 2 | 2 | 2 | 2 | 2 | 2 | 2 | 2 |
| | Stearic acid | 0.5 | 0.5 | 0.5 | 0.5 | 0.5 | 0.5 | 0.5 | 0.5 |
| | Behenic acid | 0.5 | 0.5 | 0.5 | 0.5 | 0.5 | 0.5 | 0.5 | 0.5 |
| | Stearyl alcohol | 0.3 | 0.3 | 0.3 | 0.3 | 0.3 | 0.3 | 0.3 | 0.3 |
| | Behenyl alcohol | 1.2 | 1.2 | 1.2 | 1.2 | 1.2 | 1.2 | 1.2 | 1.2 |
| | Hyaluronic acid | 0.1 | | | | | | | |
| | (Ammonium | | | | | | | | |
| | acryloyldimethyltaurate/beheneth-25 | | 0.2 | | | | | | |
| | methacrylate) crosspolymer (*2) | | | | | | | | |
| | Carboxyvinyl polymer | | | 0.2 | | | | | |
| | (Dimethyl acrylamide/sodium | | | | | | | | |
| | acryloyldimethyl taurate) | | | | 0.6 | | | | |
| | crosspolymer | | | | | | | | |
| | Stearoxy hydroxypropyl methyl | | | | | | | | |
| | cellulose (*3) | | | | | 0.25 | | | |
| | PEG90M | | | | | | 0.1 | | |
| | Xanthan gum | | | | | | | 0.1 | |
| | Hydroxy ethyl cellulose | | | | | | | | 0.1 |
| (C) | Polyoxyethylene cetyl ether (*4) | 0.2 | 0.2 | 0.2 | 0.2 | 0.2 | 0.2 | 0.2 | 0.2 |
| | EDTA-2Na•2H2O | Suitable | Suitable | Suitable | Suitable | Suitable | Suitable | Suitable | Suitable |
| | | amount | amount | amount | amount | amount | amount | amount | amount |
| | Ethanol | 3 | 3 | 3 | 3 | 3 | 3 | 3 | 3 |
| Total | | 100 | 100 | 100 | 100 | 100 | 100 | 100 | 100 |
| | Appearance | B | B | B | B | A | A | A | A |
| | Evaluation of viscosity | c | c | c | c | A | B | B | B |

From these results, it was found that with the combination of the commonly used thickener and the nonionic surfactant, the viscosity of the ultramicroemulsion was not sufficient and the appearance was deteriorated.

On the other hand, it was found that with the combination of the stearoxy hydroxypropyl methyl cellulose (B) and the nonionic surfactant (C), the viscosity could be increased while maintaining the appearance.

Next, the present inventors investigated on the blending amount of the stearoxy hydroxypropyl methyl cellulose (B).

TABLE 2

| | | Test examples | | | | |
|---|---|---|---|---|---|---|
| | | 2-1 | 2-2 | 2-3 | 2-4 | 2-5 |
| (A) | Ion-exchanged water | To 100 | To 100 | To 100 | To 100 | To 100 |
| | Glycerin | 8 | 8 | 8 | 8 | 8 |
| | 1,3-Butylene glycol | 4 | 4 | 4 | 4 | 4 |
| | Sodium stearoyl glutamate | 0.3 | 0.3 | 0.3 | 0.3 | 0.3 |
| | Potassium hydroxide | Suitable | Suitable | Suitable | Suitable | Suitable |
| | | amount | amount | amount | amount | amount |
| | Hydrogenated polyisobutene (*1) | 3 | 3 | 3 | 3 | 3 |
| | Pentaerythrityl tetraethylhexanoate | 2 | 2 | 2 | 2 | 2 |
| | Stearic acid | 0.5 | 0.5 | 0.5 | 0.5 | 0.5 |
| | Behenic acid | 0.5 | 0.5 | 0.5 | 0.5 | 0.5 |
| | Stearyl alcohol | 0.3 | 0.3 | 0.3 | 0.3 | 0.3 |
| | Behenyl alcohol | 1.2 | 1.2 | 1.2 | 1.2 | 1.2 |
| (B) | Stearoxy hydroxypropyl methyl | | 0.1 | 0.2 | 0.3 | 0.8 |
| | cellulose (*3) | | | | | |
| | EDTA-2Na•2H2O | Suitable | Suitable | Suitable | Suitable | Suitable |
| | | amount | amount | amount | amount | amount |
| | Ethanol | 3 | 3 | 3 | 3 | 3 |
| (C) | Polyoxyethylene cetyl ether (*4) | 0.4 | 0.3 | 0.3 | 0.3 | 0.3 |
| Total | | 100 | 100 | 100 | 100 | 100 |
| Evaluation | Viscosity | 20(B) | 265(B) | 6180(A) | 21650(3) | 46600(B) |
| | Appearance | A | A | A | A | A |
| | pH | 8.04 | 8.02 | 8.01 | 8.03 | 8.07 |

From these results, it was found that when the content of the stearoxy hydroxypropyl methyl cellulose (B) is in the range of 0.2% by mass or more, the oil-in-water emulsion cosmetic having a high viscosity while satisfactorily maintaining its appearance can be obtained.

Next, the present inventors investigated on the blending amount of the nonionic surfactant (C).

TABLE 3

| | | Test examples | | | | |
|---|---|---|---|---|---|---|
| | | 3-1 | 3-2 | 3-3 | 3-4 | 3-5 |
| (A) | Ion-exchanged water | To 100 | To 100 | To 100 | To 100 | To 100 |
| | Glycerin | 8 | 8 | 8 | 8 | 8 |
| | 1,3-Butylene glycol | 4 | 4 | 4 | 4 | 4 |
| | Sodium stearoyl glutamate | 0.3 | 0.3 | 0.3 | 0.3 | 0.3 |
| | Potassium hydroxide | Suitable | Suitable | Suitable | Suitable | Suitable |
| | | amount | amount | amount | amount | amount |
| | Hydrogenated polyisobutene (*1) | 3 | 3 | 3 | 3 | 3 |
| | Pentaerythrityl tetraethylhexanoate | 2 | 2 | 2 | 2 | 2 |
| | Stearic acid | 0.5 | 0.5 | 0.5 | 0.5 | 0.5 |
| | Behenic acid | 0.5 | 0.5 | 0.5 | 0.5 | 0.5 |
| | Stearyl alcohol | 0.3 | 0.3 | 0.3 | 0.3 | 0.3 |
| | Behenyl alcohol | 1.2 | 1.2 | 1.2 | 1.2 | 1.2 |
| (B) | Stearoxy hydroxypropyl methyl | 0.25 | 0.25 | 0.25 | 0.25 | 0.25 |
| | cellulose (*3) | | | | | |
| | EDTA-2Na•2H2O | Suitable | Suitable | Suitable | Suitable | Suitable |
| | | amount | amount | amount | amount | amount |
| | Ethanol | 3 | 3 | 3 | 3 | 3 |
| (C) | Polyoxyethylene cetyl ether (*4) | 0.1 | 0.2 | 0.6 | 0.8 | 1 |
| Total | | 100 | 100 | 100 | 100 | 100 |
| Evaluation | Viscosity | 2110(A) | 8370(A) | 8620(A) | 2660(A) | 2000(A) |
| | Appearance | A | A | A | A | A |
| | pH | 8.08 | 8.04 | 8.08 | 8.12 | 8.15 |

From these results, it was found that when the blending ratio of the nonionic surfactant (C) to the stearoxy hydroxypropyl methyl cellulose (B) is in the range of 1:0.3 to 1:4, the oil-in-water emulsion cosmetic having a high viscosity while satisfactorily maintaining its appearance can be obtained.

Next, the present inventors investigated on the type of nonionic surfactant (C).

TABLE 4

| | | Test Examples | | | | | | | |
|---|---|---|---|---|---|---|---|---|---|
| | | 4-1 | 4-2 | 4-3 | 4-4 | 4-5 | 4-6 | 4-7 | 4-8 |
| (A) | Ion-exchanged water | To 100 | To 100 | To 100 | To 100 | To 100 | To 100 | To 100 | To 100 |
| | Glycerin | 8 | 8 | 8 | 8 | 8 | 8 | 8 | 8 |
| | 1,3-Butylene glycol | 4 | 4 | 4 | 4 | 4 | 4 | 4 | 4 |
| | Sodium stearoyl glutamate | 0.3 | 0.3 | 0.3 | 0.3 | 0.3 | 0.3 | 0.3 | 0.3 |
| | Potassium hydroxide | Suitable | Suitable | Suitable | Suitable | Suitable | Suitable | Suitable | Suitable |
| | | amount | amount | amount | amount | amount | amount | amount | amount |
| | Hydrogenated polyisobutene (*1) | 3 | 3 | 3 | 3 | 3 | 3 | 3 | 3 |
| | Pentaerythrityl tetraethylhexanoate | 2 | 2 | 2 | 2 | 2 | 2 | 2 | 2 |
| | Stearic acid | 0.5 | 0.5 | 0.5 | 0.5 | 0.5 | 0.5 | 0.5 | 0.5 |
| | Behenic acid | 0.5 | 0.5 | 0.5 | 0.5 | 0.5 | 0.5 | 0.5 | 0.5 |
| | Stearyl alcohol | 0.3 | 0.3 | 0.3 | 0.3 | 0.3 | 0.3 | 0.3 | 0.3 |
| | Behenyl alcohol | 1.2 | 1.2 | 1.2 | 1.2 | 1.2 | 1.2 | 1.2 | 1.2 |
| (B) | Stearoxy hydroxypropyl methyl | | | | | | | | |
| | cellulose (*3) | | 0.25 | 0.25 | 0.25 | 0.25 | 0.25 | 0.25 | 0.25 |
| | EDTA-2Na•2H2O | | Suitable | Suitable | Suitable | Suitable | Suitable | Suitable | Suitable |
| | | | mount | amount | amount | amount | amount | amount | amount |
| | Ethanol | | 3 | 3 | 3 | 3 | 3 | 3 | 3 |
| (C) | Polyoxyethylene cetyl ether (*4) | | | 0.4 | | | | | |
| | POE hydrogenated castor oil (*5) | | | | 0.4 | | | | |
| | Polyoxyethylene glyceryl isostearate | | | | | 0.4 | | | |
| | (*6) | | | | | | | | |
| | Polyoxyethylene phytosterol (*7) | | | | | | 0.4 | | |
| | Polyoxyethylene behenyl ether (*8) | | | | | | | 0.4 | |
| | Polyoxyethylene sorbitan | | | | | | | | |
| | monostearate (*9) | | | | | | | | 0.4 |
| Total | | 100 | 100 | 100 | 100 | 100 | 100 | 100 | 100 |
| Evaluation | Viscosity | 23 | 460 | 9510 | 6820 | 10100 | 7380 | 10700 | 2560 |
| | | B | B | A | A | B | A | B | A |
| | Appearance | A | B | A | A | A | A | A | A |
| | pH | 7.85 | 7.93 | 7.98 | 7.96 | 7.96 | 7.97 | 8.01 | 8.01 |

(*5) NIKKOL ® HCO-60 (manufactured by Nikko Chemicals Co., Ltd.)
(*6) EMALEX ® GWIS-160N (manufactured by Nihon Emulsion Co., Ltd.)
(*7) NIKKOL ® BPS-30 (manufactured by Nikko Chemicals Co., Ltd)
(*8) NIKKOL ® BB-20 (manufactured by Nikko Chemicals Co., Ltd.)
(*9) NIKKOL ® TS-10V (manufactured by Nikko Chemicals Co., Ltd.)

From these test examples, it was found that various nonionic surfactants (C) increase the viscosity. Particularly, with the hydrophilic nonionic surfactant, the oil-in-water emulsion cosmetic having preferable appearance and viscosity was obtained.

Hereinafter, the present inventors further describe formulation examples of the oil-in-water emulsion cosmetic according to the present invention.

TABLE 5

| | Formulation | Formulation | Formulation |
|---|---|---|---|
| | example 1 | example 2 | example 3 |
| Water | To 100 | To 100 | To 100 |
| Glycerin | 8 | 5 | 5 |
| 1,3-Butylene glycol | 4 | 8 | 8 |
| Polyoxyethylene(14)polyoxy- | 1 | 1 | 1 |
| propylene(7)dimethyl | | | |
| ether | | | |
| Sodium stearoyl glutamate | 0.3 | 0.3 | 0.3 |
| Caustic potash | Suitable | Suitable | Suitable |
| | amount | amount | amount |
| Mineral oil | 3 | 3 | 3 |
| Pentaerythrityl | 2 | 2 | 5 |
| tetraethylhexanoate | | | |
| Stearic acid | 0.5 | 0.5 | 0.5 |
| Behenic acid | 0.5 | 0.5 | 0.5 |
| Stearyl alcohol | 0.3 | 0.3 | 0.3 |
| Behenyl alcohol | 1.2 | 1.2 | 1.2 |
| Stearoxy hydroxypropyl | 0.25 | 0.25 | 0.25 |
| methyl cellulose (*3) | | | |
| EDTA-2Na | Suitable | Suitable | Suitable |
| | amount | amount | amount |
| Ethanol | 3 | 3 | 3 |
| Polyoxyethylene | 0.2 | 0.2 | 0.2 |
| cetyl ether (*4) | | | |
| Tranexamic acid | 2 | — | — |
| Dipotassium glycyrrhizinate | 0.05 | — | — |
| Nicotinamide | — | 5 | — |
| Tocopherol acetate | — | 0.5 | — |
| Water, phenoxyethanol, | — | 0.1 | — |
| saccharomyces culture lysate | | | |
| Allantoin | — | 0.1 | — |
| Polysorbate 20, BHT, retinol | — | — | 0.1 |
| Sunflower seed oil, | — | — | 0.1 |
| BHT, retinol acetate | | | |
| Stearyl glycyrrhetinate | — | — | 0.05 |

1. An oil-in-water emulsion cosmetic, comprising:
(A) 10 to 50% by mass of an ultramicroemulsion comprising
(a1) an anionic surfactant, and
(a2) a linear higher alcohol having at least 16 or more carbon atoms that can form an α-gel in water in combination with (a1);
(B) 0.2 to 2% by mass of a stearoxy hydroxypropyl methyl cellulose having the following formula (1); and
(C) 0.1 to 1% by mass of a nonionic surfactant, 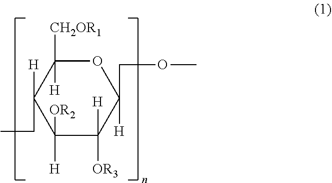
wherein R₁, R₂ and R₃ is at least one selected from a group consisting of —[CH₂CH(CH₃)O]x-R₄, —[CH₂CH₂O]y-R₄ and —[CH₂CH(OH)CH₂O]z-R₄ where x, y, and z are identical or different, and represent an integer of 0 to 4; and
R₄ represents one or more groups selected from a hydrogen atom, a linear or branched alkyl group having 1 to 4 carbon atoms, and a linear alkyl group having 10 to 28 carbon atoms; at least one of R₄ is a linear alkyl group having 10 to 28 carbon atoms; the linear alkyl group having 10 to 28 carbon atoms is preferably a linear alkyl group having 12 to 22 carbon atoms; and
n represents an integer of 200 to 200000.

2. The oil-in-water emulsion cosmetic, according to claim 1, wherein:
a viscosity of said cosmetic is 2000 to 100000 mPa·s.

3. The oil-in-water emulsion cosmetic, according to claim 1, wherein:
the ultramicroemulsion (A) is composed of oil droplets having an average particle size of 5 nm to 300 nm.

4. The oil-in-water emulsion cosmetic, according to claim 2, wherein:
the ultramicroemulsion (A) is composed of oil droplets having an average particle size of 5 nm to 300 nm.
